(19) United States
(12) Patent Application Publication
(10) Pub. No.: US 2019/0102514 A1
(43) Pub. Date: Apr. 4, 2019

(54) GENETIC VARIANT IDENTIFICATION FOR COMPLEX DISEASE

(71) Applicant: INTERNATIONAL BUSINESS MACHINES CORPORATION, Armonk, NY (US)

(72) Inventors: Laxmi P. PARIDA, YORKTOWN HEIGHTS, NY (US); Daniel E. PLATT, YORKTOWN HEIGHTS, NY (US)

(21) Appl. No.: 15/801,694

(22) Filed: Nov. 2, 2017

Related U.S. Application Data
(63) Continuation of application No. 15/724,377, filed on Oct. 4, 2017.

(51) Int. Cl. G06F 19/22; G06F 19/18; C12Q 1/6874; G01N 33/48

(52) U.S. Cl. CPC G06F 19/22; G06F 19/18; G01N 33/48; C12Q 1/6874

(57) ABSTRACT

Embodiments of the present invention are directed to a computer-implemented method for generating a list of genetic variants. A non-limiting example of the computer-implemented method includes receiving genetic and biological data. The exemplary method also includes generating data patterns from the genetic and biological data with data mining. The method also includes determining redescription distances between each of a plurality of data patterns. The method also includes generating computational homology filtrations from the redescription distances using a topological data analysis and homology groups including homology group elements based upon the computational homology filtrations. The method also includes generating a single nucleotide polymorphism combination list based upon the homology group elements and redescription clusters.

### DOMESTIC AND/OR FOREIGN PRIORITY

This application is a continuation of U.S. application Ser. No. 15/724,337, titled “Genetic Variant Identification for Complex Disease” filed Oct. 4, 2017, the contents of which are incorporated by reference herein in its entirety.

### BACKGROUND

The present invention generally relates to characterizing genetic data, and more specifically, to genetic variant identification for complex disease.

The quantity and availability of genetic and other biological system data is increasing. Examining and comparing genetic information and phenotypes across large populations can potentially reveal useful information for treatment and identification of disease patterns, causes, and risks. For instance, genetic data can be organized several different ways including, for example, around pathways, systems, gene-gene interactions including epistatic interactions, gene-environment interactions including metabiome interactions, and environment-driven epigenetic changes. Evidence of complex associations and causative factors, such as gene-gene and gene-environment interactions, can exist within the large sets of genetic and biological systems data currently available. For example, complex diseases can involve gene-environment interactions that could be identified within a relevant set of data including the pertinent genetic and environmental clues. However, such interactions could in some cases require relatively large genetic and environmental data sets for the relevant association to arise. The availability of genetic and other biological system data is increasing and, along with it, the potential to discover and interpret complex diseases and the basis for such diseases.

### SUMMARY

Embodiments of the present invention are directed to a computer-implemented method for generating a list of genetic variants. A non-limiting example of the computer-implemented method includes receiving, to a processor, genetic and biological data. The exemplary method also includes using data mining to generate, by the processor, data patterns from the genetic and biological data. The method also includes determining, by the processor, redescription distances between each of a plurality of data patterns, wherein the redescription distances are based upon a dissimilarity between lists of samples identified by each data pattern in the plurality of data patterns. The method also includes generating, by the processor, redescription clusters by applying single-linkage agglomerative binary clustering to the redescription distances. The method also includes generating, by the processor, computational homology filtrations from the redescription distances using a topological data analysis and homology groups including homology group elements based upon the computational homology filtrations. The method also includes using a discriminant analysis to generate, by the processor, a single nucleotide polymorphism combination list based upon the homology group elements and redescription clusters.

Embodiments of the present invention are directed to a computer program product for generating a list of genetic variants. A non-limiting example of the computer program product includes a computer readable storage medium readable by a processing circuit and storing program instructions for execution by the processing circuit for performing a method. The method includes receiving genetic and biological data. The exemplary method also includes using data mining to generate data patterns from the genetic and biological data. The exemplary method also includes determining, by the processor, redescription distances between each of a plurality of data patterns, wherein the redescription distances are based upon a dissimilarity between lists of samples identified by each data pattern in the plurality of data patterns. The exemplary method also includes generating redescription clusters of the patterns by applying single-linkage agglomerative binary clustering to the redescription distances. The exemplary method also includes generating computational homology filtrations from the redescription distances by using a topological data analysis and homology groups comprising homology group elements based upon the computational homology filtrations. The method also includes using a discriminant analysis to generate a genetic variant list based upon the homology group elements and redescription clusters.

Embodiments of the invention are directed to a processing system for generating a list of genetic variants, the processing system including a processor in communication with one or more types of memory. The processor can be configured to receive genetic and biological data including data items. The processor can also be configured to use data mining to generate data patterns from the genetic and biological data. The processor can also be configured to determine redescription distances between each of a plurality of data patterns, wherein the redescription distances are based upon a dissimilarity between lists of samples identified by each data pattern in the plurality of data patterns. The processor can also be configured to generate redescription clusters by applying single-linkage agglomerative binary clustering to the redescription distances. The processor can also be configured to generate computational homology filtrations from the redescription distances by using a topological data analysis and homology groups comprising homology group elements based upon the computational homology filtrations. The processor can also be configured to use a discriminant analysis to generate a single nucleotide polymorphism list based upon the homology group elements and redescription clusters.

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 depicts a block diagram illustrating one example of a processing system for practice of the teachings herein;

FIG. 2 depicts a flow diagram of a method according to one or more embodiments of the present invention;

FIG. 3 depicts an exemplary system according to one or more embodiments of the present invention.

The diagrams depicted herein are illustrative. There can be many variations to the diagram or the operations described therein without departing from the spirit of the invention. For instance, the actions can be performed in a differing order or actions can be added, deleted or modified. Also, the term “coupled” and variations thereof describes having a communications path between two elements and does not imply a direct connection between the elements with no intervening elements/connections between them. All of these variations are considered a part of the specification.

In the accompanying figures and following detailed description of the disclosed embodiments, the various elements illustrated in the figures are provided with two or three digit reference numbers. With minor exceptions, the leftmost digit(s) of each reference number correspond to the figure in which its element is first illustrated.

### DETAILED DESCRIPTION

Various embodiments of the invention are described herein with reference to the related drawings. Alternative embodiments of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” may be understood to include any integer number greater than or equal to one, i.e. one, two, three, four, etc. The terms “a plurality” may be understood to include any integer number greater than or equal to two, i.e. two, three, four, five, etc. The term “connection” may include both an indirect “connection” and a direct “connection.”

The terms “about,” “substantially,” “approximately,” and variations thereof, are intended to include the degree of error associated with measurement of the particular quantity based upon the equipment available at the time of filing the application. For example, “about” can include a range of ±8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

Turning now to an overview of technologies that are more specifically relevant to aspects of the invention, availability of genetic and other biological system data has increased exponentially over the past decade. The dimensionality of genetic and other biological system data can be organized around pathways, systems, gene-gene interactions with epistatic interactions, gene-environment interactions and environmental changes, for example. Genetic data can potentially reveal a variety of information useful for understanding biological pathways and systems.

Genome wide association studies (GWAS) can be used to analyze or identify relevant genetic associations in organisms. GWAS can be used, for instance, to examine one or more genetic variants in a set of individuals to determine whether the variants are associated with particular traits. GWAS, for example, can potentially identify associations between genetic variants, such as single nucleotide polymorphisms (SNPs), and traits associated with human diseases or disorders. The types of information revealed by GWAS, however, can be limited. For instance, although GWAS can sometimes identify SNPs associated with traits in major human disease based on observable phenotype, GWAS can fail to reveal other types of relationships such as relevant interactions between an SNP and another SNP.

The large quantities of genetic and biological systems data available and involved in analyzing relevant interactions and associations poses challenges to revealing relevant and useful patterns and interactions. Mining tools have potential to reveal evidence of relevant genetic associations and relationships. However, the large quantities of available data, and the large quantities of data that can be required to reveal complex relationships, pose challenges and can render conventional mining techniques unfeasible or inadequate.

Discriminant approaches, such as Linear Discriminant Analysis (LDA), can seek to determine a linear combination of features associated with an event or other variable. In context of genetic data analysis, for instance, LDA could potentially identify linear combinations of genetic variants, gene expression results, and other biological data that maximize the variance between groups marked by composite phenotypes. However, discriminant approaches can lead to relatively large increases in data volume, which can be problematic and/or unworkable in the context of large initial data sets such as those associated with genome-wide genetic analysis. Thus, determining genetic basis for metabolic pathways or compound phenotypes in large genetic and biological systems data sets with conventional discriminant approaches can be unworkable. Thus, while conventional systems could sometimes associate an observable phenotype, such as hypertension, with a subset of SNPs from principal component analysis (PCA) of a limited data set, identification of complex associations that might be revealed in a larger set of genetic and biological systems data, such as the genetic basis for metabolic pathways or for phenotypes associated with combinations of SNPs, can be unattainable.

Turning now to an overview of the aspects of the invention, one or more embodiments of the invention address the above-described shortcomings of the prior art by providing systems that can analyze genetic and biological systems data to evaluate complex diseases. Embodiments of the invention can include PCA to facilitate discriminant analysis, such as LDA, to identify relevant genetic variants, such as SNPs, and/or their combinations, from relatively large data sets. Embodiments of the invention can generate relevant genetic variants associated with complex diseases and phenotypes by including pattern discovery to identify significant patterns, identifying significant redescriptions within the patterns, and then applying a discriminant analysis to reveal important discriminant variants.

The above-described aspects of the invention address the shortcomings of the prior art by probing interactions within networks and systems that include genetic and biological systems data, including relatively large data sets to probe gene-gene and gene-environment interactions and other higher order associations to characterize and identify SNPs associated with disease. Embodiments of the invention can determine a genetic basis for metabolic pathways. Some embodiments of the invention can identify a genetic basis for compound phenotypes. Embodiments of the invention can include subsets of identified discriminating SNPs to provide a smaller data set to mine for significance. In some embodiments of the invention, the identified discriminating SNPs can be further evaluated with GWAS studies to further characterize genetic associations. Embodiments of the invention can advantageously boost the determined significance of SNPs leading to increased reliability in genetic association identifications. Some embodiments of the invention can enhance GWAS studies to provide greater specificity among leading genes.

The aforementioned results can be achieved by performing PCA and singular value decomposition (SVD) with descriptors or compound phenotypes to identify an orthogonal basis that spans the sampled data space and provides a representation of the dataset in that basis, and yet is small enough to perform a discriminant analysis, such as LDA, to identify a discriminating subset of SNPs. Such methodologies are an improvement over conventional genome-wide assessments because they facilitate discriminant approaches that would otherwise be precluded because of the large volumes of input data.

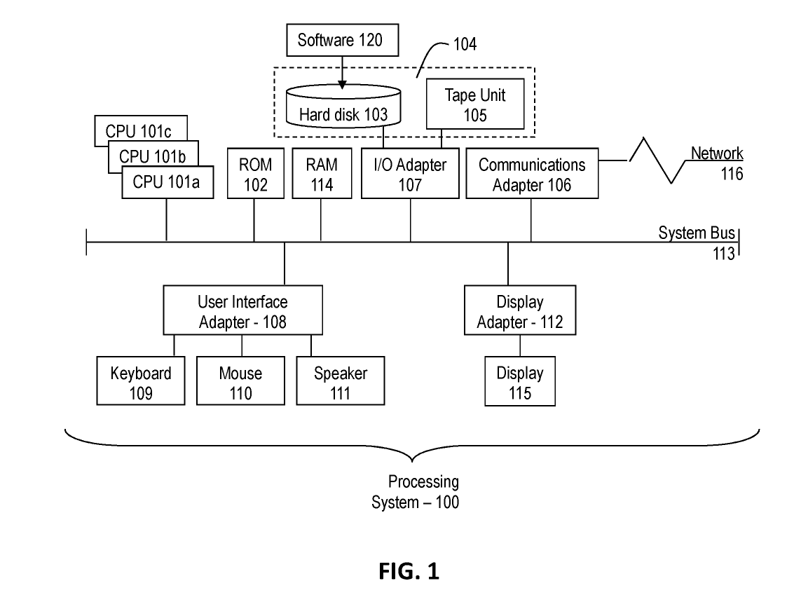

Turning now to a more detailed description of aspects of the invention, FIG. 1 depicts an embodiment of a processing system 100 for implementing the teachings herein. In this embodiment of the system 100, there is provided one or more central processing units (processors) 101a, 101b, 101c, etc. (collectively or generically referred to as processor(s) 101). In one embodiment, each processor 101 can include a reduced instruction set computer (RISC) microprocessor. Processors 101 are coupled to system memory 114 and various other components via a system bus 113. Read only memory (ROM) 102 is coupled to the system bus 113 and can include a basic input/output system (BIOS), which controls certain basic functions of system 100.

FIG. 1 further depicts an input/output (I/O) adapter 107 and a network adapter 106 coupled to the system bus 113. I/O adapter 107 can be a small computer system interface (SCSI) adapter that communicates with a hard disk 103 and/or tape storage drive 105 or any other similar component. I/O adapter 107, hard disk 103, and tape storage device 105 are collectively referred to herein as mass storage 104. Operating system 120 for execution on the processing system 100 can be stored in mass storage 104. A network adapter 106 interconnects bus 113 with an outside network 116 enabling data processing system 100 to communicate with other such systems. A screen (e.g., a display monitor) 115 is connected to system bus 113 by display adaptor 112, which can include a graphics adapter to improve the performance of graphics intensive applications and a video controller. In one embodiment, adapters 107, 106, and 112 can be connected to one or more I/O busses that are connected to system bus 113 via an intermediate bus bridge (not shown). Suitable I/O buses for connecting peripheral devices such as hard disk controllers, network adapters, and graphics adapters typically include common protocols, such as the Peripheral Component Interconnect (PCI). Additional input/output devices are shown as connected to system bus 113 via user interface adapter 108 and display adapter 112. A keyboard 109, mouse 110, and speaker 111 all interconnected to bus 113 via user interface adapter 108, which can include, for example, a Super I/O chip integrating multiple device adapters into a single integrated circuit.

In exemplary embodiments of the invention, the processing system 100 includes a graphics processing unit 130. Graphics processing unit 130 is a specialized electronic circuit designed to manipulate and alter memory to accelerate the creation of images in a frame buffer intended for output to a display. In general, graphics processing unit 130 is very efficient at manipulating computer graphics and image processing, and has a highly parallel structure that makes it more effective than general-purpose CPUs for algorithms where processing of large blocks of data is done in parallel.

Thus, as configured in FIG. 1, the system 100 includes processing capability in the form of processors 101, storage capability including system memory 114 and mass storage 104, input means such as keyboard 109 and mouse 110, and output capability including speaker 111 and display 115. In one embodiment, a portion of system memory 114 and mass storage 104 collectively store an operating system such as the AIX® operating system from IBM Corporation to coordinate the functions of the various components shown in FIG. 1.

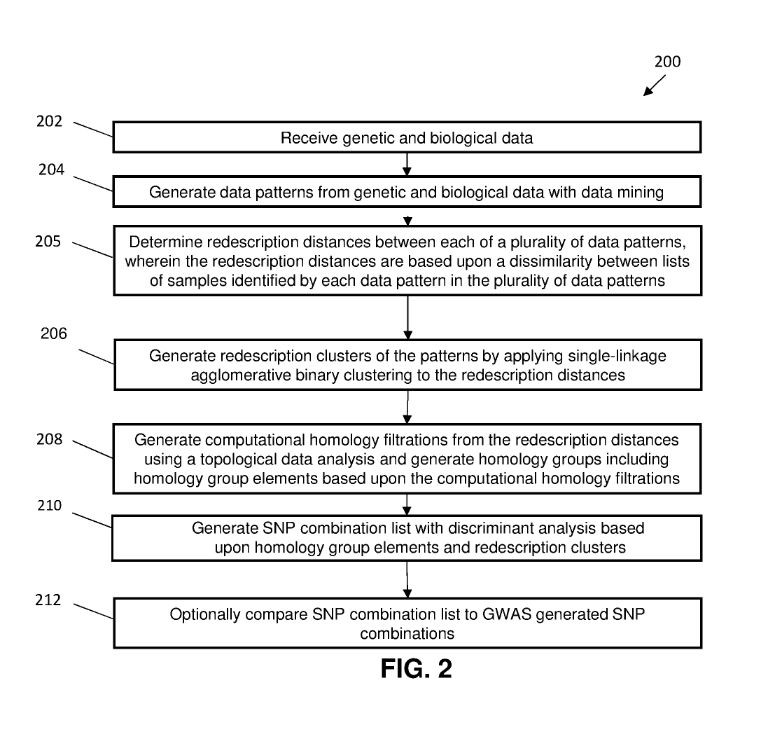

Continuing with the more detailed description of aspects of the present invention, FIG. 2 depicts a method 200 of genetic variant list generation according to embodiments of the invention. The method 200 includes, as shown at block 202, receiving genetic and biological data. The method 200 can also include, as shown at block 204, generating data patterns from genetic and biological systems data with data mining. The method 200 can also include, as shown at block 205, determining redescription distances between each of a plurality of data patterns, wherein the redescription distances are based upon a dissimilarity between lists of samples identified by each data pattern in the plurality of data patterns, for example as measured by Jaccard distances. The method 200 can also include, as shown at block 206, generating redescription clusters of the patterns by applying single-linkage agglomerative binary clustering to the redescription distances. The method 200 can also include, as shown at block 208, generating computational homology filtrations from the redescription distances using a topological data analysis and generating homology groups including homology group elements based upon the computational homology filtrations. The method 200 can also include, as shown at block 210, generating a genetic variant or SNP combination list based upon by the homology group elements and redescription clusters, including SNPs distinguished by the homology group elements and redescription clusters. The method 200 also optionally includes comparing the SNP combination list to GWAS-generated or GWAS-styled SNP combinations, as shown at block 212.

The genetic data can include genomic or metagenomic data for an individual, for a subset of individuals, or for one or more populations including, for example, genetic variant data and/or single nucleotide polymorphism data. Biological data can include, for example, medical data, such as medical diagnoses, medical history, and medication lists, phenotypes, demographic data, environmental data concerning a biological system, biological pathway data, metabolic data, biochemical data, or any other data that can potentially be correlated with a biological or genetic condition or state. The genetic and biological data can include data entries, such as “subjects” that include study subjects or enrollees in a study, “conditions” such as hypertension, diabetes or coronary artery disease, and the like.

Data mining can include any known method capable of selecting patterns according to statistical significance. Data mining can include pattern discovery methods, including selecting patterns according to statistical significance. Data mining can implement, for example, logistic regression. In some embodiments of the invention, data mining includes assigning a measure of significance to each pattern association, wherein the measure of significance quantifies the chance that a pattern association would be observed among samples as a result of random variation.

Redescriptions and redescription clusters, including statistically significant redescriptions and redescription clusters, can be identified or within or generated from the mined data, for example, through redescription mining. For example, data mining of medical data can generate a plurality of subsets of data including medical descriptors such as diabetes, hypertension, coronary heart disease, obesity, and cancer, to name a few. Redescription mining can include analyzing subsets of data associated with one or more descriptors that can be amenable to multiple different descriptions and generating appropriate redescriptions for such subsets based upon the analysis. Statistically significant redescriptions can identify composite phenotypes and logical relationships among phenotypes marking underlying biological processes associated by Jaccard or similar distances. Such biological processes can include, for instance, biological pathways, epistatic interactions, gene-environment interactions, and the like. Redescription can identify logical equivalence and construct logical relationships between variables.

For example, given a set of data entries, patterns can be generated that reveal groups or subsets of subjects that share certain patterns (e.g., hypertension=T & diabetes=T & coronary artery disease=F). Each such pattern can be associated with a list of data entries or subjects that share the pattern.

Distances, such as Jaccard distances, can be determined between such lists of data entries. In some embodiments of the invention, clusters can be defined based upon groups of items sharing at least one neighbor nearer than a given distance threshold. Such clusters can form equivalence classes, wherein all the patterns in a given cluster are assumed to be equivalent. For example, in the above exemplary scenario, if the Diabetic=T list, and Diabetic=T and Hypertension=T list are both in a cluster and have a high degree of similarity (i.e., few disagreements in their respective lists), then D=D & H such that D implies H (the diabetes list is a subset of the hypertension list). Such a result could reveal, for example, that diabetics tend to be hypertensive. Because, in the above exemplary scenario, Diabetes=T results in nearly the same list as Diabetes=T & Hypertension=T, both of these patterns capture the same list and can be said to “redescribe” that list. Equivalent patterns are referred to herein as “redescriptions.”

TDA computes homology groups generated through analysis of redescription distances among patterns. In some embodiments of the invention, TDA determines how the redescription clusters of patterns are related in topological space and can identify or distinguish between related and/or unrelated groups. Generation of homology groups can include identifying combinations in the mined data that are likely to be related for further analysis. In some embodiments of the invention, TDA is performed on calculated distances between lists of data entries sharing a pattern. In some embodiments of the invention, TDA can reveal relationships between and among patterns.

LDA computation can be performed by singular value decomposition (SVD), based on PCA with descriptors or compound phenotypes. SVD, for example, can recode the whole space spanned by the sample data, advantageously reducing the computational size of the mined data set in some embodiments of the invention, enabling downstream LDA analysis.

For example, redescription could reveal that most diabetics have hypertension. TDA can provide more information among related logical statements that redescription discovers, including some evidence of possible missing relationships (holes) in the data. These patterns can identify groups of data that share patterns (redescriptions), implying logical relationships among the patterns, and which form topological relationships among the patterns. Each of these groups of data represent augmented or compound phenotypes that reflect distinctive biological processes subject to the logical relationships among the data, e.g. “most type 2 diabetics have hypertension.” Such augmented phenotypes can then show distinct genetic relationships among them that can be probed using GWAS or LDA. This can give distinctive genetic signatures marking specific biological pathways marked by the redescription clusters.

Some embodiments of the invention include performing a discriminant analysis. Discriminant analysis can include, for example, LDA, support vector machine (SVM) approaches, or similar analysis. Discriminant analysis can be applied to homology groups alone or in combination with other data, such as redescription clusters. Discriminant analyses can be applied to homology groups, genetic data, gene expression data, and the like and can maximize the variation of linear combinations of genetic markers between versus within groups identified by the redescription clusters and homology groups. In some embodiments of the invention, discriminant analysis yields a genetic variant list including linear combinations, most important discriminant variants, and/or statistical significance for the linear combinations, such as p-values. In some embodiments of the invention, linear combinations can include SNP or gene combinations such as gene-gene combinations, gene-environment combinations, or gene-phenotype combinations.

Some embodiments of the invention include performing GWAS or a GWAS-style measure. Methods of performing such studies and measures are known. For example, one embodiment of the invention can include a genome wide comparison of a healthy set of individuals to a subset of individuals having a certain characteristic, such as a disease. In some embodiments of the invention, GWAS includes analysis of linear combinations from an SNP combination list generated in a discriminant analysis.

In some embodiments of the invention, GWAS is performed on the same data set used to generate an SNP combination list according to embodiments of the invention and an SNP list generated by the GWAS analysis is compared to the SNP combination list. Based upon the comparison, some embodiments of the invention can include generating a refined SNP list including, for example, a subset of the SNP combination list that is also included on the SNP list generated by the GWAS analysis.

Embodiments of the invention can identify connections between phenotypes and multiple SNPs or other genetic variants.

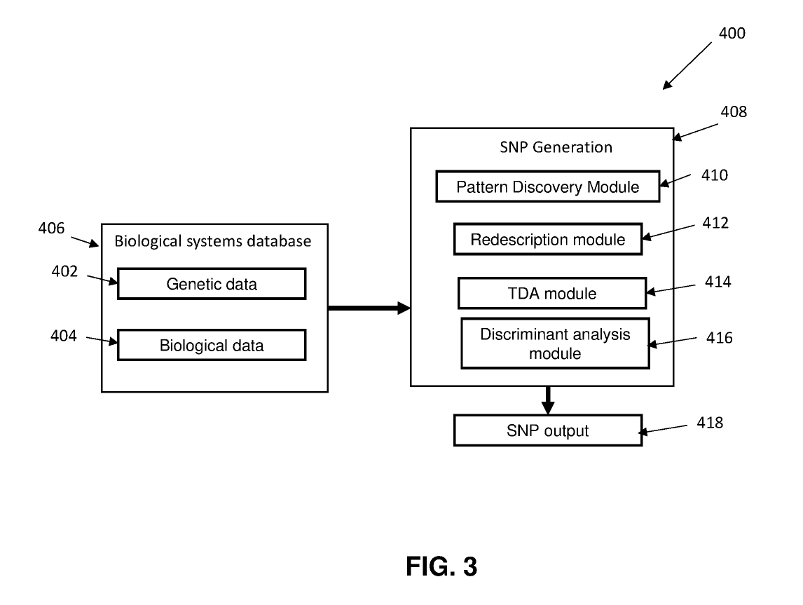

FIG. 3 depicts an exemplary system 400 for SNP identification. The system 400 can include a biological systems database 406 including, for example, genetic data 402 and biological data 404. The system 400 can also include an SNP generation module 408 in communication with the biological systems database 406. In some embodiments of the invention, the SNP generation module 408 can include a pattern discovery module 410. The pattern discovery module 410 can mine genetic and biological data to identify statistically significant patterns. The SNP generation module 408 can also include a redescription module 412. The redescription module 412 can, for instance, identify statistically significant redescriptions within the data to generate composite phenotypes and logical relationships among phenotypes marking underlying biological processes associated by Jaccard distances. The SNP generation module 408 can also include a TDA module 414. The TDA module 414 can, for example, construct homology groups from the Jaccard distances generated by the redescription module 412. The SNP generation module 408 can also include a discriminant analysis module 416. The discriminant analysis module 416 can apply a discriminant approach, including LDA, GWAS, and/or other discriminant methods, to the data to generate individual, or linear combinations of the most important discriminant variants, and to quantify significance. The system 400 can also include an SNP output 418. The SNP output can include, for instance, a user interface, such as a display, for providing linear combinations, most important discriminant variants, significance and/or related data generated by the SNP Generation module 408 to a user.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may include copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages, languages supported on virtual environments such as Java or Python, or interpreted computational environments, such as R. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instruction by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein includes an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which includes one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A computer-implemented method for generating a list of genetic variants comprising:
receiving, to a processor, genetic and biological data;
using data mining to generate, by the processor, data patterns from the genetic and biological data;
determining, by the processor, redescription distances between each of a plurality of data patterns, wherein the redescription distances are based upon a dissimilarity in lists of samples identified by each data pattern in the plurality of data patterns;
generating, by the processor, redescription clusters of the patterns by applying single-linkage agglomerative binary clustering to the redescription distances;
generating, by the processor, computational homology filtrations from the redescription distances using a topological data analysis and homology groups including homology group elements based upon the computational homology filtrations; and
using a discriminant analysis to generate, by the processor, a genetic variant list based upon the homology group elements and redescription clusters.

2. The computer-implemented method of claim 1, wherein the genetic variant list comprises a gene-gene interaction.

3. The computer-implemented method of claim 1, wherein the genetic variant list comprises a gene-environment interaction.

4. The computer-implemented method of claim 1, further comprising conducting a genome wide association study (GWAS) on the genetic and biological data to generate a GWAS-single nucleotide polymorphism (SNP) combination list.

5. The computer-implemented method of claim 1, wherein the discriminant analysis comprises a linear discriminant analysis.

6. The computer-implemented method of claim 1, wherein the discriminant analysis comprises support vector machines analysis.

7. The computer-implemented method of claim 1, wherein the genetic variant list comprises a list of most important discriminant variants.
